(19) United States
(12) Patent Application Publication
(10) Pub. No.: US 2026/0027233 A1
(43) Pub. Date: Jan. 29, 2026

(54) GJA1-20K COMPOSITIONS AND METHODS FOR MITIGATING ISCHEMIA-REPERFUSION INJURY

(71) Applicant: UNIVERSITY OF UTAH RESEARCH FOUNDATION, Salt Lake City, UT (US)

(72) Inventors: Guillaume Hoareau, Salt Lake City, UT (US); Robin M. Shaw, Salt Lake City, UT (US)

(21) Appl. No.: 18/998,041

(22) PCT Filed: Aug. 31, 2023
(86) PCT No.: PCT/US2023/073223

§ 371 Date: Jan. 23, 2025

Related U.S. Application Data
(60) Provisional application No. 63/402,724, filed on Aug. 31, 2022.
(60) Provisional application No. 63/437,586, filed on Jan. 6, 2023.

(51) Int. Cl. A61K 48/00; A61K 9/00; A61K 38/00; A61P 9/10; C07K 14/47; C12N 15/86

(52) U.S. Cl. CPC A61K 48/005; A61K 9/0019; A61K 48/0075; A61P 9/10; C07K 14/4702; C12N 15/86; A61K 38/00; C07K 2319/20; C12N 2750/14143

(57) ABSTRACT

Described herein are compositions and methods for treating, preventing, reducing the likelihood of having, reducing the severity of and/or slowing the progression of ischemia-reperfusion injury in a subject using GJA1-20k as a therapeutic agent. In one embodiment, the compositions and methods comprise a GJA1-20k peptide therapy. In another embodiment, the compositions and methods comprise a GJA1-20k gene therapy. In some embodiments, the GJA1-20k therapy comprises truncated forms of GJA1-20k. In some embodiments, the GJA1-20k therapy mitigates mitochondrial stress and dysfunction.

### CROSS-REFERENCE TO RELATED APPLICATIONS

This application is a U.S. national phase application of International Patent Application No. PCT/US2023/073223, filed Aug. 31, 2023, which_claims priority to U.S. Provisional Patent Application Nos. 63/437,586, filed on Jan. 6, 2023, and 63/402,724, filed on Aug. 31, 2022, each of which is incorporated by reference herein in its entirety.

### FEDERALLY SPONSORED RESEARCH

This invention was made with government support under HT9425-24-1-0140 awarded by the Defense Health Agency, Medical Research and Development Branch, andgrants HL155886 and HL138577 awarded by the National Institutes of Health. The government has certain rights in this invention.

### REFERENCE TO SEQUENCE LISTING

This application was filed with a Sequence Listing XML in ST.26 XML format in accordance with 37 C.F.R. § 1.821. The Sequence Listing XML file submitted in the USPTO Patent Center, “026389-0006-WO01_sequence_listing_XML_25 Aug. 2023.xml,” was created on Aug. 25, 2023, contains 4 sequences, has a file size of 6.0 Kbytes, and is hereby incorporated by reference in its entirety into the specification.

### TECHNICAL FIELD

Described herein are compositions and methods for treating, preventing, reducing the likelihood of having, reducing the severity of and/or slowing the progression of ischemia-reperfusion injury in a subject using GJA1-20k as a therapeutic agent. In one embodiment, the compositions and methods comprise a GJA1-20k peptide therapy. In another embodiment, the compositions and methods comprise a GJA1-20k gene therapy. In some embodiments, the GJA1-20k therapy comprises truncated forms of GJA1-20k. In some embodiments, the GJA1-20k therapy mitigates mitochondrial stress and dysfunction.

### BACKGROUND

Ischemia-reperfusion injury, which is the tissue damage caused when blood supply returns to tissue following a period of ischemia or lack of oxygen, exacerbates cellular injury, dysfunction, and ultimately death. This syndrome is a serious condition that can arise in several medical situations including severe bleeding, hemorrhagic and hypovolemic shock, solid organ transplant, cardiac bypass surgery, cardiac angioplasty, radio-opaque dye injury to kidneys, damage to downstream organs in vascular surgery, and many others. In these situations, it is crucial to protect cell and organ function. Specifically, mitochondria are essential mediators of ischemia-reperfusion injury, and mitochondrial damage and dysfunction can potentiate ischemia-reperfusion injury.

Currently, there are no established medical adjuncts to effectively treat and/or prevent the damaging effects of ischemia-reperfusion injury, and patients that have suffered traumatic injuries leading to ischemia or ischemia-reperfusion injury cannot benefit from the protective effects of ischemic preconditioning. Some previous studies have investigated active cooling; however, these cooling methods are impractical and not applicable to all situations, such as traumatic injuries. Rather, the cooling techniques that have been tested primarily relate to the specific fields of cardiac bypass and cardiac arrest as a means to limit neurological injury after a catastrophic cardiac event.

Thus, what is needed are new compositions and methods for mitigating mitochondrial stress and dysfunction to treat and prevent ischemia-reperfusion injury in patients.

### SUMMARY

One embodiment described herein is a method of treating, preventing, reducing the likelihood of having, reducing the severity of, and/or slowing the progression of ischemia-reperfusion injury in a subject, the method comprising: administering to the subject a therapeutically effective amount of a pharmaceutical composition comprising: a GJA1-20k polypeptide or functional variant or fragment thereof; or a GJA1-20k gene expression vector comprising a polynucleotide sequence encoding a GJA1-20k polypeptide or functional variant or fragment thereof. In one aspect, the pharmaceutical composition is administered to the subject by intravenous (IV) injection. In another aspect, the GJA1-20k polypeptide or functional variant or fragment thereof comprises an amino acid sequence having at least 90-99% identity to any one of SEQ ID NO: 1 or 3. In another aspect, the GJA1-20k polypeptide or functional variant or fragment thereof comprises an amino acid sequence selected from any one of SEQ ID NO: 1 or 3. In another aspect, the GJA1-20k polypeptide or functional variant or fragment thereof further comprises a polyaspartate (D10) peptide tag (SEQ ID NO: 2). In another aspect, the polynucleotide sequence has at least 90-99% identity to SEQ ID NO: 4. In another aspect, the polynucleotide sequence is SEQ ID NO: 4. In another aspect, the GJA1-20k gene expression vector is selected from a viral vector, an adeno-associated virus (AAV) vector, a recombinant AAV (rAAV) vector, a single-stranded AAV vector, a double-stranded AAV vector, a self-complementary AAV (scAAV) vector, or combinations thereof. In another aspect, the GJA1-20k gene expression vector is an AAV vector of a serotype selected from AAV1, AAV2, AAV3, AAV4, AAV5, AAV6, AAV7, AAV8, AAV9, AAV10, AAV11, AAV12, or a hybrid serotype thereof. In another aspect, the GJA1-20k gene expression vector is an AAV9 vector. In another aspect, the ischemia-reperfusion injury in the subject is the result of severe bleeding, hemorrhagic or hypovolemic shock, resuscitative endovascular balloon occlusion of the aorta, solid organ transplant, cardiac bypass surgery, cardiac angioplasty, radio-opaque dye injury to kidneys, damage to downstream organs in vascular surgery, or combinations thereof.

Another embodiment described herein is a pharmaceutical composition comprising a GJA1-20k polypeptide or functional variant or fragment thereof. In one aspect, the GJA1-20k polypeptide or functional variant or fragment thereof comprises an amino acid sequence having at least 90-99% identity to any one of SEQ ID NO: 1 or 3. In another aspect, the GJA1-20k polypeptide or functional variant or fragment thereof comprises an amino acid sequence selected from any one of SEQ ID NO: 1 or 3. In another aspect, the GJA1-20k polypeptide or functional variant or fragment thereof further comprises a polyaspartate (D10) peptide tag (SEQ ID NO: 2).

Another embodiment described herein is a pharmaceutical composition comprising a GJA1-20k gene expression vector comprising a polynucleotide sequence encoding a GJA1-20k polypeptide or functional variant or fragment thereof. In one aspect, the polynucleotide sequence has at least 90-99% identity to SEQ ID NO: 4. In another aspect, the polynucleotide sequence is SEQ ID NO: 4. In another aspect, the GJA1-20k gene expression vector is selected from a viral vector, an adeno-associated virus (AAV) vector, a recombinant AAV (rAAV) vector, a single-stranded AAV vector, a double-stranded AAV vector, a self-complementary AAV (scAAV) vector, or combinations thereof. In another aspect, the GJA1-20k gene expression vector is an AAV vector of a serotype selected from AAV1, AAV2, AAV3, AAV4, AAV5, AAV6, AAV7, AAV8, AAV9, AAV10, AAV11, AAV12, or a hybrid serotype thereof. In another aspect, the GJA1-20k gene expression vector is an AAV9 vector.

Another embodiment described herein is the use of a GJA1-20k peptide therapy in a medicament for treating, preventing, reducing the likelihood of having, reducing the severity of, and/or slowing the progression of ischemia-reperfusion injury in a subject. In one aspect, the GJA1-20k peptide therapy comprises a therapeutically effective amount of a pharmaceutical composition comprising: a GJA1-20k polypeptide or functional variant or fragment thereof.

Another embodiment described herein is the use of a GJA1-20k gene therapy in a medicament for treating, preventing, reducing the likelihood of having, reducing the severity of, and/or slowing the progression of ischemia-reperfusion injury in a subject. In one aspect, the GJA1-20k gene therapy comprises a therapeutically effective amount of a pharmaceutical composition comprising: a GJA1-20k gene expression vector comprising a polynucleotide sequence encoding a GJA1-20k polypeptide or functional variant or fragment thereof.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the urine-to-serum concentration levels of neutrophil gelatinase-associated lipocalin (NGAL) in control-treated and GJA1-20k peptide-treated pigs at the end of a six-hour experiment.

FIG. 2A-B show the serum concentrations of various cytokines over time in control-treated and GJA1-20k peptide-treated pigs. FIG. 2A shows a heatmap of the log 2 fold change for serum protein expression levels of 16 different cytokines in control-treated and GJA1-20k peptide-treated pigs at 105 min, 180 min, and 360 min from T-0. FIG. 2B shows the serum concentration levels (pMol/mL) for interleukin-6 (IL-6, top) and interferon-gamma (IFNg, bottom) in control-treated and GJA1-20k peptide-treated pigs at 0 min, 105 min, 180 min, and 360 min. *=p-value <0.05.

### DETAILED DESCRIPTION

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “gene,” “nucleic acid,” “nucleotide,” “polynucleotide,” “oligonucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein. Nucleic acids may be single stranded or double stranded or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

As used herein, “variants” may include, but are not limited to, those that include conservative amino acid substitution(s), truncated variants, single nucleotide polymorphism (SNP) variants, degenerate variants, and biologically active portions of a gene. A “degenerate variant” as used herein refers to a variant that has a mutated nucleotide sequence, but still encodes the same polypeptide due to the redundancy of the genetic code. There are 20 naturally occurring amino acids; however, some of these share similar characteristics. For example, leucine and isoleucine are both aliphatic, branched, and hydrophobic. Similarly, aspartic acid and glutamic acid are both small and negatively charged. Conservative substitutions in proteins often have a smaller effect on function than non-conservative mutations. Although there are many ways to classify amino acids, they are often sorted into six main groups on the basis of their structure and the general chemical characteristics of their R groups. A mutation among the same class of amino acids is considered a conservative amino acid substitution.

The term “functional” when used in conjunction with “variant” or “fragment” refers to an entity or molecule which possess a biological activity that is substantially similar to a biological activity of the entity or molecule of which it is a variant or fragment thereof. In accordance with the present invention, a GJA1-20k polypeptide may be modified, for example, to facilitate or improve identification, expression, isolation, storage and/or administration, so long as such modifications do not reduce its function to an unacceptable level. In various embodiments, a GJA1-20k polypeptide functional variant or fragment thereof has at least 10%, 20%, 30%, 40%, 50%, 60%, 70%, 80%, 90%, 95%, or 99% of the function of a full-length wildtype GJA1-20k isoform.

As used herein, “substantial identity” of polynucleotide sequences means that a polynucleotide comprises a sequence that has at least 25% sequence identity compared to a reference sequence as determined using programs known in the art (e.g., Basic Local Alignment Search Tool (BLAST)). In preferred embodiments, percent identity can be any integer from 25% to 100%. More preferred embodiments include polynucleotide sequences that have at least about: 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, or 99% sequence identity compared to a reference sequence. These values can be appropriately adjusted to determine corresponding identity of proteins encoded by two nucleotide sequences by taking into account codon degeneracy, amino acid similarity, reading frame positioning, and the like. Accordingly, polynucleotides of the present invention encoding a protein or polypeptide of the present invention include nucleic acid sequences that have substantial identity to the nucleic acid sequences that encode the proteins or polypeptides of the present invention. Polynucleotides encoding a polypeptide comprising an amino acid sequence that has at least about: 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, or 99% sequence identity compared to a reference polypeptide sequence are also preferred.

As used herein, “substantial identity” of amino acid sequences (and of polypeptides having these amino acid sequences) means that an amino acid sequence comprises a sequence that has at least 25% sequence identity compared to a reference sequence as determined using programs known in the art (e.g., BLAST). In preferred embodiments, percent identity can be any integer from 25% to 100%. More preferred embodiments include amino acid or polypeptide sequences that have at least about: 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, or 99% sequence identity compared to a reference sequence. Polypeptides that are “substantially identical” share amino acid sequences except that residue positions which are not identical may differ by one or more conservative amino acid changes, as described above. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Exemplary conservative amino acid substitution groups include valine-leucine-isoleucine, phenylalanine-tyrosine, lysine-arginine, alanine-valine, aspartic acid-glutamic acid, and asparagine-glutamine. Accordingly, polypeptides or proteins, encoded by the polynucleotides of the present invention, include amino acid sequences that have substantial identity to the amino acid sequences of the reference polypeptide sequences.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to +10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.” All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to +10% of any value within the range or within 3 or more standard deviations, including the end points.

As used herein, the terms “active ingredient” or “active pharmaceutical ingredient” refer to a pharmaceutical agent, active ingredient, compound, or substance, compositions, or mixtures thereof, that provide a pharmacological, therapeutic, often beneficial, effect. In some embodiments, disclosed compositions may further comprise one or more pharmaceutically acceptable carriers or excipients. Example carriers may include, but are not limited to, liposomes, polymeric micelles, microspheres, microparticles, dendrimers, nanoparticles, and the like. As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the term “administering” refers to the placement of an agent or a composition as disclosed herein into a subject by a method or route which results in at least partial localization of the agents or composition at a desired site. “Route of administration” may refer to any administration pathway known in the art, including but not limited to oral, intravenous (IV), topical, aerosol, nasal, via inhalation, anal, intra-anal, peri-anal, transmucosal, transdermal, parenteral, enteral, or local. “Parenteral” refers to a route of administration that is generally associated with injection, including intracranial, intraventricular, intrathecal, epidural, intradural, intraorbital, infusion, intracapsular, intracardiac, intradermal, intramuscular, intraperitoneal, intrapulmonary, intraspinal, intrasternal, intrathecal, intrauterine, intravascular, intravenous (IV), intraarterial, subarachnoid, subcapsular, subcutaneous, transmucosal, or transtracheal. Via the parenteral route, the agent or composition may be in the form of solutions or suspensions for IV infusion or IV injection, or as lyophilized powders. Via the enteral route, the agent or composition can be in the form of capsules, gel capsules, tablets, sugar-coated tablets, syrups, suspensions, solutions, powders, granules, emulsions, microspheres or nanospheres or lipid vesicles or polymer vesicles allowing controlled release. Via the topical route, the agent or composition can be in the form of aerosol, lotion, cream, gel, ointment, suspensions, solutions or emulsions. In one embodiment, the agent or composition may be provided in a powder form and mixed with a liquid, such as water, to form a beverage. In accordance with the present invention, “administering” can be self-administering. For example, it is considered “administering” when a subject consumes a composition as disclosed herein.

As used herein, “contacting” refers to contacting a target cell with an agent (e.g., a GJA1-20k polypeptide or gene expression vector) using any method that is suitable for placing the agent on, in, or adjacent to a target cell. For example, when the cells are in vitro, contacting the cells with the agent can comprise adding the agent to culture medium containing the cells. For example, when the cells are in vivo, contacting the cells with the agent can comprise administering the agent to a subject.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments. In some embodiments of the present invention, a subject is in need of treatment if the subject is suffering from, or at risk of suffering from, ischemia and/or ischemia-reperfusion injury.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic manner. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest. In one embodiment of the present invention, a method of treating, preventing, reducing the likelihood of having, reducing the severity of, and/or slowing the progression of ischemia-reperfusion injury in a subject is described.

As used herein, “sample” or “target sample” refers to any sample in which the presence and/or level of a target analyte or target biomarker is to be detected or determined. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological or bodily fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

As used herein, “target analyte” or “target biomarker” refers to a substance that is associated with a biological state or a biological process, such as a disease state or a diagnostic or prognostic indicator of a disease or disorder (e.g., an indicator identifying the likelihood of the existence or later development of a disease or disorder). The presence or absence of a biomarker, or the increase or decrease in the concentration of a biomarker, can be associated with and/or be indicative of a particular state or process. Biomarkers can include, but are not limited to, cells or cellular components (e.g., a viral cell, a bacterial cell, a fungal cell, a cancer cell, etc.), small molecules, lipids, carbohydrates, nucleic acids, peptides, proteins, enzymes, antigens, and antibodies. A biomarker can be derived from an infectious agent, such as a bacterium, fungus or virus, or can be an endogenous molecule that is found in greater or lesser abundance in a subject suffering from a disease or disorder as compared to a healthy individual (e.g., an increase or decrease in expression of a gene or gene product).

Connexin 43 (Cx43) is a gap junction protein that allows intercellular communication and also regulates cell proliferation, differentiation, and death. The GJA1 gene encodes the full-length Cx43 protein (i.e., the longest isoform of GJA1-43k). It has previously been reported that N-terminally truncated Cx43 isoforms exist endogenously in the heart, and that they are produced by alternative translation of the GJA1 mRNA to generate various shorter isoforms including GJA1-32k, GJA1-29k, GJA1-26k, GJA1-20k, GJA1-11k, and GJA1-7k. The GJA1-20k isoform is 20 kDa and is the most abundant Cx43 isoform. GJA1-20k is an internally-translated variant that is effectively an N-terminal truncation of the Cx43 protein, consisting of the C-terminus without the transmembrane domains of Cx43. GJA1-20k is a stress response protein that protects mitochondrial function and mitigates mitochondrial stress.

Described herein are GJA1-20k-based compositions and methods for treating, preventing, reducing the likelihood of having, reducing the severity of, and/or slowing the progression of ischemia-reperfusion injury in a subject. As described herein, a pharmaceutical composition comprising a GJA1-20k peptide or gene therapy may be administered to a subject before or after ischemia or ischemia-reperfusion has occurred to protect against ischemic and ischemia-reperfusion injury in the subject. In some embodiments, the GJA1-20k peptide or gene therapy may treat or prevent ischemia-reperfusion injury in the subject through mitigating mitochondrial stress and dysfunction. In some embodiments, the GJA1-20k peptide or gene therapy may also mitigate renal injury in the subject.

The GJA1-20k-based compositions and methods described herein may provide particular advantages over the existing means known in the art for treating or preventing ischemia-reperfusion injury. For example, patients that have experienced sudden traumatic injuries leading to ischemia or ischemia-reperfusion injury are currently unable to benefit from the protective effects of ischemic preconditioning. However, the GJA1-20k therapies described herein offer a solution to this problem. In particular, the GJA1-20k peptide therapy provides an option for prompt administration of a peptide having biological activity that ultimately recapitulates preconditioning for these subjects suffering from traumatic injury. A GJA1-20k peptide as described herein could be injected at the point of traumatic injury until transport to an advanced resuscitation platform, which would be especially relevant for rural or military scenarios.

In addition, some patients with serious injuries to the abdomen may require occlusion of the aorta. A GJA1-20k peptide as described herein could be injected to mitigate ischemia-reperfusion in those patients. Furthermore, complete aortic occlusion times are limited due to profound ischemia-reperfusion injury. GJA1-20k peptide administration may allow for longer occlusion periods, which would also be pertinent for rural or military scenarios. Additionally, a GJA1-20k peptide as described herein could potentially be more potent than the current cardioplegia solutions used to preserve tissue for transplant and also to protect the heart when stopped for cardiac surgery (with the patient on cardio-pulmonary bypass). The peptide therapy could also be used prophylactically in clinical situations of anticipated ischemia such as major cardiac surgery, coronary artery procedures, major vascular surgery, or use of toxic agents such as radiopaque dye in angiography and imaging procedures.

A GJA1-20k gene therapy as described herein may provide a more prophylactic approach to precondition the subject for any potential traumatic injury that could occur. The GJA1-20k gene therapy would be especially beneficial to subjects having a higher likelihood for sudden traumatic injury, such as high-risk military personnel. The GJA1-20k gene therapy approach could also be used prophylactically in clinical situations to benefit subjects preparing for major cardiac or vascular surgeries by preconditioning these subjects prior to surgery.

In some embodiments, gene therapy may be used to deliver GJA1-20k to individuals at risk for ischemia-reperfusion injury (e.g., military personnel) or patients before anticipated ischemia-reperfusion injury. This GJA1-20k gene therapy approach would confer those individuals with an ischemic-preconditioning type of metabolic protection.

Various embodiments of the present invention provide a pharmaceutical composition comprising a GJA1-20k peptide therapy or a GJA1-20k gene therapy for treating or preventing ischemia-reperfusion injury in a subject. In various embodiments, the GJA1-20k peptide or gene is of a mammal. In various embodiments, the GJA1-20k peptide or gene is of a primate, for example, a human, a chimpanzee, a gorilla, or a monkey. In various embodiments, the GJA1-20k peptide or gene is of a horse, a goat, a donkey, a cow, a bull, or a pig. In various embodiments, the GJA1-20k peptide or gene is of a rodent, for example, a mouse, a rat, or a guinea pig. In various embodiments, the GJA1-20k peptide or gene is of a chicken, a duck, a frog, a dog, a cat, or a rabbit.

### Compositions

The present disclosure provides GJA1-20k peptide therapy pharmaceutical compositions comprising a GJA1-20k polypeptide or functional variant or fragment thereof. The present disclosure also provides pharmaceutical compositions comprising a GJA1-20k gene expression vector comprising a polynucleotide sequence encoding a GJA1-20k polypeptide or functional variant or fragment thereof. The disclosed compositions may further comprise one or more pharmaceutically acceptable carriers or excipients.

### GJA1-20k Polypeptides

The disclosed pharmaceutical compositions may comprise a GJA1-20k polypeptide or functional variant or fragment thereof. As used herein, the term “GJA1-20k polypeptide or functional variant or fragment thereof” refers to any polypeptide having an amino acid sequence translated from the C-terminal portion of the GJA1 gene, or a functional variant or fragment of that amino acid sequence. In some embodiments, the GJA1-20k polypeptide or functional variant or fragment thereof comprises a truncated GJA1-20k polypeptide.

The GJA1-20k polypeptide may be synthesized and purified using techniques and methods known in the art. For example, the GJA1-20k polypeptide may be synthesized in yeast (e.g., Pichia pastoris) or other suitable host organisms known in the art. In some embodiments, the GJA1-20k polypeptide or functional variant or fragment thereof comprises the human full-length wildtype (WT) GJA1-20k polypeptide sequence (amino acids are numbered according to the full-length human Cx43; that is, the 170 amino acid human full-length GJA1-20k sequence is amino acids 213-382 of human Cx43; SEQ ID NO: 1):

| SEQ ID NO: 1 | | |
|---|---|---|
| Human Full-length Wildtype GJA1-20k Amino Acid Sequence | | |
| 213 | MLVVSLVSLALNIIELFYVFFKGVKDRVKGKSDPYHATSGALSPAKDCGSQKY | 265 |
| 266 | AYFNGCSSPTAPLSPMSPPGYKLVTGDRNNSSCRNYNKQASEQNWANYSA | 315 |
| 316 | EQNRMGQAGSTISNSHAQPEDFPDDNQNSKKLAAGHELQPLAIVDQRPSSR | 366 |
| 367 | ASSRASSRPRPDDLEI | 382 |

In various embodiments, the GJA1-20k polypeptide can be modified for better production, storage, administration, detection, delivery efficiency, etc. For example, in one embodiment, the GJA1-20k polypeptide can be modified with one or more molecular tags and/or linkers. As another example, the GJA1-20k polypeptide can be codon optimized for expression in bacteria and/or yeast. As another example, the GJA1-20k polypeptide can be PEGylated for better stability, better solubility, reduced antigenicity, reduced renal clearance, and prolonged circulatory time. As another example, the GJA1-20k polypeptide can be modified with one or more cell penetrating peptides (CPP). As yet another example, the GJA1-20k polypeptide can be modified with one or more polyaspartate (D10) peptide tags, which aid in biochemical handling (SEQ ID NO: 2):

SEQ ID NO: 2
Polyaspartate (D10) Peptide Tag Sequence
DDDDDDDDDD

In various embodiments, the GJA1-20k polypeptide may comprise one or more mutations from the full-length wildtype GJA1-20k form. For example, in various embodiments, the GJA1-20k polypeptide functional variants are variants with one or more conservative amino acid substitutions, where the variant retains a substantial amount of biological activity. Exemplary conservative amino acid substitution groups include, but are not limited to, valine-leucine-isoleucine, phenylalanine-tyrosine, lysine-arginine, alanine-valine, aspartic acid-glutamic acid, and asparagine-glutamine.

In some embodiments, the GJA1-20k polypeptide comprises one or more mutations and has at least about 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, or 99% sequence homology to the wildtype GJA1-20k polypeptide while still retaining a substantial amount of biological activity. For example, the mutated GJA1-20k polypeptide amino acid sequence may have greater than about 75%, greater than about 80%, greater than about 90%, greater than about 95%, or greater than about 99% sequence homology to the wildtype GJA1-20k polypeptide while still retaining a substantial amount of biological activity.

In some embodiments, the GJA1-20k polypeptide may be a truncated variant (N-terminal and/or C-terminal truncation) from the full-length wildtype GJA1-20k form. For example, in one non-limiting embodiment, the GJA1-20k polypeptide is truncated to remove an N-terminal transmembrane region, where the polypeptide comprises amino acids 236-382 of full-length human Cx43 (“GJA1-20k-dTM”). In some embodiments, this N-terminal truncation that removes the N-terminal transmembrane domain may be important for improving GJA1-20k polypeptide synthesis and/or stability. In one non-limiting exemplary embodiment, the GJA1-20k polypeptide comprises 180 amino acids comprising an N-terminal poly-His tag, a linker, the truncated GJA1-20k-dTM fragment, and a C-terminal D10 peptide tag (SEQ ID NO: 3):

SEQ ID NO: 3
Exemplary Truncated GJA1-20k Polypeptide Sequence
MHHHHHHGGGGSGGGGSVKDRVKGKSDPYHATSGALSPAKDCGSQKYAYF
NGCSSPTAPLSPMSPPGYKLVTGDRNNSSCRNYNKQASEQNWANYSAEQN
RMGQAGSTISNSHAQPFDFPDDNQNSKKLAAGHELQPLAIVDQRPSSRAS
SRASSRPRPDDLEIGGSGGSDDDDDDDDDD

In some embodiments, the GJA1-20k polypeptide is a truncated variant and has at least about 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, or 99% sequence homology to the wildtype GJA1-20k polypeptide while still retaining a substantial amount of biological activity. For example, the truncated GJA1-20k polypeptide amino acid sequence may have greater than about 75%, greater than about 80%, greater than about 90%, greater than about 95%, or greater than about 99% sequence homology to the wildtype GJA1-20k polypeptide while still retaining a substantial amount of biological activity.

### GJA1-20k Polynucleotides and Gene Expression Vectors

The disclosed pharmaceutical compositions may comprise a GJA1-20k gene expression vector comprising a polynucleotide sequence encoding a GJA1-20k polypeptide or functional variant or fragment thereof.

In some embodiments, various gene expression vectors as described herein are used to produce various GJA1-20k polypeptides or functional variants or fragments thereof. For example, various gene expression vectors are introduced into bacteria or yeast to produce various GJA1-20k polypeptides or functional variants thereof, which are later isolated. In various embodiments, the gene expression vector is a plasmid. In various embodiments, the gene expression vector is a viral vector, adeno-associated virus (AAV) vector, recombinant AAV (rAAV) vector, single-stranded AAV vector, double-stranded AAV vector, self-complementary AAV (scAAV) vector, or a combination thereof. In various embodiments, the gene expression vector is a polynucleotide or a virus particle. In various embodiments, the serotype of the virus particle is AAV1, AAV2, AAV3, AAV4, AAV5, AAV6, AAV7, AAV8, AAV9, AAV10, AAV11, AAV12, or a hybrid serotype thereof. In one embodiment, the GJA1-20k gene expression vector is an AAV9 vector containing a CMV promoter, wherein a polynucleotide sequence encoding a GJA1-20k polypeptide or functional variant or fragment thereof is cloned into the AAV9 vector.

In various embodiments, the GJA1-20k gene expression vector comprising a polynucleotide sequence encoding a GJA1-20k polypeptide or functional variant or fragment thereof can be modified for better expression, production, storage, administration, detection, delivery efficiency, etc. In various embodiments, the GJA1-20k gene expression vector may comprise one or more molecular tags and/or linkers. In one embodiment, the GJA1-20k gene expression vector comprises polynucleotide sequences encoding an N-terminal V5 epitope tag and a C-terminal linker. In another embodiment, the GJA1-20k gene expression vector comprises a polynucleotide sequence encoding a C-terminal GFP tag.

In various embodiments, the polynucleotide sequence encoding a GJA1-20k polypeptide or functional variant or fragment thereof may comprise one or more mutations. For example, in various embodiments, the GJA1-20k polynucleotide sequence encodes GJA1-20k polypeptide functional variants having one or more conservative amino acid substitutions, where the variant retains a substantial amount of biological activity. In another example, the polynucleotide sequence includes one or more mutations to prevent the expression of one or more GJA1 isoforms other than GJA1-20k, such as GJA1-11k and/or GJA1-7k. In one aspect, the GJA1-20k polynucleotide sequence includes one or more point mutations to generate a GJA1-20k polypeptide having a M281L mutation (with respect to SEQ ID NO: 1). This M281L mutation specifically prevents the translation of the shorter GJA1-11k and GJA1-7k isoforms. In one exemplary embodiment, the GJA1-20k polynucleotide sequence comprises 534 nucleotides encoding an N-terminal poly-His tag and the full-length GJA1-20k polypeptide sequence having the M281L mutation (SEQ ID NO: 4):

SEQ ID NO: 4
Exemplary GJA1-20k Polynucleotide Sequence
atgcatcatcaccatcaccacatgctggtggtgtccttggtgtccctggc
cttgaatatcattgaactcttctatgttttcttcaagggcgttaaggatc
gggttaagggaaagagcgacccttaccatgcgaccagtggtgcgctgagc
cctgccaaagactgtgggtctcaaaaatatgcttatttcaatggctgctc
ctcaccaaccgctcccctctcgcctctatctcctcctgggtacaagctgg
ttactggcgacagaaacaattcttcttgccgcaattacaacaagcaagca
agtgagcaaaactgggctaattacagtgcagaacaaaatcgactagggca
ggcgggaagcaccatctctaactcccatgcacagccttttgatttccccg
atgataaccagaattctaaaaaactagctgctggacatgaattacagcca
ctagccattgtggaccagcgaccttcaagcagagccagcagtcgtgccag
cagcagacctcggcctgatgacctggagatctag

### Methods of Treatment

The present disclosure also provides a method of treating, preventing, reducing the likelihood of having, reducing the severity of, and/or slowing the progression of ischemia-reperfusion injury in a subject. The method comprises: administering to the subject a therapeutically effective amount of a pharmaceutical composition comprising: a GJA1-20k polypeptide or functional variant or fragment thereof, as described herein; or a GJA1-20k gene expression vector comprising a polynucleotide sequence encoding a GJA1-20k polypeptide or functional variant or fragment thereof, as described herein.

In various embodiments, the pharmaceutical composition comprising the GJA1-20k polypeptide or functional variant or fragment thereof may be administered as a therapeutic agent (e.g., GJA1-20k peptide therapy) to a subject to treat, prevent, reduce the likelihood of having, reduce the severity of, and/or slow the progression of the ischemia-reperfusion injury. In various embodiments, the pharmaceutical composition comprising the GJA1-20k gene expression vector comprising a polynucleotide sequence encoding a GJA1-20k polypeptide or functional variant or fragment thereof may be administered as a therapeutic agent (e.g., GJA1-20k gene therapy) to a subject to treat, prevent, reduce the likelihood of having, reduce the severity of, and/or slow the progression of the ischemia-reperfusion injury.

In various embodiments, the ischemia-reperfusion injury in the subject may result from conditions including, but not limited to, severe bleeding, hemorrhagic or hypovolemic shock, resuscitative endovascular balloon occlusion of the aorta (REBOA), solid organ transplant, cardiac bypass surgery, cardiac angioplasty, radio-opaque dye injury to kidneys, damage to downstream organs in vascular surgery, or combinations thereof.

In various embodiments, the subject is a human. In various embodiments, the subject is a mammalian subject including but not limited to human, monkey, ape, dog, cat, cow, horse, goat, pig, rabbit, mouse, and rat.

In various embodiments, the pharmaceutical composition comprising a GJA1-20k polypeptide or functional variant or fragment thereof or a GJA1-20k gene expression vector comprising a polynucleotide sequence encoding a GJA1-20k polypeptide or functional variant or fragment thereof is administered to the subject by intravenous (IV) injection. Pharmaceutical compositions as described herein may also be administered using alternative routes, including but not limited to intravascular, intraarterial, intramuscular, subcutaneous, intraperitoneal, aerosol, nasal, via inhalation, oral, transmucosal, transdermal, parenteral, implantable pump or reservoir, continuous infusion, enteral application, topical application, local application, capsules and/or injections.

In some embodiments, the subject is administered a single dose of the disclosed pharmaceutical compositions. In other embodiments, the subject is administered a plurality of doses of the disclosed pharmaceutical compositions over a period of time. For example, in various nonlimiting embodiments, a pharmaceutical composition as described herein may be administered to a subject once a day (SID/QD), twice a day (BID), three times a day (TID), four times a day (QID), or more, or continuously, so as to administer a therapeutically effective amount of the pharmaceutical composition to the subject, where the therapeutically effective amount is any one or more of the doses described herein. In some embodiments, a pharmaceutical composition as described herein is administered to a subject 1-3 times per day, 1-7 times per week, 1-9 times per month, 1-12 times per year, or more. In other embodiments, a pharmaceutical composition as described herein is administered for about 1-10 days, 10-20 days, 20-30 days, 30-40 days, 40-50 days, 50-60 days, 60-70 days, 70-80 days, 80-90 days, 90-100 days, 1-6 months, 6-12 months, 1-5 years, or more. In various embodiments, a pharmaceutical composition as described herein is administered at about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, 900-1000 mg/kg, or a combination thereof.

The actual dosing regimen can depend upon many factors, including, but not limited to, the judgment of a trained physician, the overall condition of the subject, and the severity of the ischemia or ischemia-reperfusion injury. The actual dosage can also depend on the determined experimental effectiveness of the specific pharmaceutical composition (e.g., a GJA1-20k polypeptide or functional variant or fragment thereof; a GJA1-20k gene expression vector comprising a polynucleotide sequence encoding a GJA1-20k polypeptide or functional variant or fragment thereof) that is administered. For example, the dosage may be determined based on in vitro responsiveness of relevant cultured cells, or in vivo responses observed in appropriate animal models or human studies for both the GJA1-20k peptide therapy and the GJA1-20k gene therapy.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:

Clause 1. A method of treating, preventing, reducing the likelihood of having, reducing the severity of, and/or slowing the progression of ischemia-reperfusion injury in a subject, the method comprising:
administering to the subject a therapeutically effective amount of a pharmaceutical composition comprising:
   a GJA1-20k polypeptide or functional variant or fragment thereof; or
   a GJA1-20k gene expression vector comprising a polynucleotide sequence encoding a GJA1-20k polypeptide or functional variant or fragment thereof.

Clause 2. The method of clause 1, wherein the pharmaceutical composition is administered to the subject by intravenous (IV) injection.

Clause 3. The method of clause 1 or 2, wherein the GJA1-20k polypeptide or functional variant or fragment thereof comprises an amino acid sequence having at least 90-99% identity to any one of SEQ ID NO: 1 or 3.

Clause 4. The method of any one of clauses 1-3, wherein the GJA1-20k polypeptide or functional variant or fragment thereof comprises an amino acid sequence selected from any one of SEQ ID NO: 1 or 3.

Clause 5. The method of any one of clauses 1-4, wherein the GJA1-20k polypeptide or functional variant or fragment thereof further comprises a polyaspartate (D10) peptide tag (SEQ ID NO: 2).

Clause 6. The method of any one of clauses 1-5, wherein the polynucleotide sequence has at least 90-99% identity to SEQ ID NO: 4.

Clause 7. The method of any one of clauses 1-6, wherein the polynucleotide sequence is SEQ ID NO: 4.

Clause 8. The method of any one of clauses 1-7, wherein the GJA1-20k gene expression vector is selected from a viral vector, an adeno-associated virus (AAV) vector, a recombinant AAV (rAAV) vector, a single-stranded AAV vector, a double-stranded AAV vector, a self-complementary AAV (scAAV) vector, or combinations thereof.

Clause 9. The method of any one of clauses 1-8, wherein the GJA1-20k gene expression vector is an AAV vector of a serotype selected from AAV1, AAV2, AAV3, AAV4, AAV5, AAV6, AAV7, AAV8, AAV9, AAV10, AAV11, AAV12, or a hybrid serotype thereof.

Clause 10. The method of any one of clauses 1-9, wherein the GJA1-20k gene expression vector is an AAV9 vector.

Clause 11. The method of any one of clauses 1-10, wherein the ischemia-reperfusion injury in the subject is the result of severe bleeding, hemorrhagic or hypovolemic shock, resuscitative endovascular balloon occlusion of the aorta, solid organ transplant, cardiac bypass surgery, cardiac angioplasty, radio-opaque dye injury to kidneys, damage to downstream organs in vascular surgery, or combinations thereof.

Clause 12. A pharmaceutical composition comprising a GJA1-20k polypeptide or functional variant or fragment thereof.

Clause 13. The pharmaceutical composition of clause 12, wherein the GJA1-20k polypeptide or functional variant or fragment thereof comprises an amino acid sequence having at least 90-99% identity to any one of SEQ ID NO: 1 or 3.

Clause 14. The pharmaceutical composition of clause 12 or 13, wherein the GJA1-20k polypeptide or functional variant or fragment thereof comprises an amino acid sequence selected from any one of SEQ ID NO: 1 or 3.

Clause 15. The pharmaceutical composition of any one of clauses 12-14, wherein the GJA1-20k polypeptide or functional variant or fragment thereof further comprises a polyaspartate (D10) peptide tag (SEQ ID NO: 2).

Clause 16. A pharmaceutical composition comprising a GJA1-20k gene expression vector comprising a polynucleotide sequence encoding a GJA1-20k polypeptide or functional variant or fragment thereof.

Clause 17. The pharmaceutical composition of clause 16, wherein the polynucleotide sequence has at least 90-99% identity to SEQ ID NO: 4.

Clause 18. The pharmaceutical composition of clause 16 or 17, wherein the polynucleotide sequence is SEQ ID NO: 4.

Clause 19. The pharmaceutical composition of any one of clauses 16-18, wherein the GJA1-20k gene expression vector is selected from a viral vector, an adeno-associated virus (AAV) vector, a recombinant AAV (rAAV) vector, a single-stranded AAV vector, a double-stranded AAV vector, a self-complementary AAV (scAAV) vector, or combinations thereof.

Clause 20. The pharmaceutical composition of any one of clauses 16-19, wherein the GJA1-20k gene expression vector is an AAV vector of a serotype selected from AAV1, AAV2, AAV3, AAV4, AAV5, AAV6, AAV7, AAV8, AAV9, AAV10, AAV11, AAV12, or a hybrid serotype thereof.

Clause 21. The pharmaceutical composition of any one of clauses 16-20, wherein the GJA1-20k gene expression vector is an AAV9 vector.

Clause 22. Use of a GJA1-20k peptide therapy in a medicament for treating, preventing, reducing the likelihood of having, reducing the severity of, and/or slowing the progression of ischemia-reperfusion injury in a subject.

Clause 23. The use of clause 22, wherein the GJA1-20k peptide therapy comprises a therapeutically effective amount of a pharmaceutical composition comprising:
a GJA1-20k polypeptide or functional variant or fragment thereof.

Clause 24. Use of a GJA1-20k gene therapy in a medicament for treating, preventing, reducing the likelihood of having, reducing the severity of, and/or slowing the progression of ischemia-reperfusion injury in a subject.

Clause 25. The use of clause 24, wherein the GJA1-20k gene therapy comprises a therapeutically effective amount of a pharmaceutical composition comprising:
a GJA1-20k gene expression vector comprising a polynucleotide sequence encoding a GJA1-20k polypeptide or functional variant or fragment thereof.

### EXAMPLES

### Example 1

### GJA1-20K Reduces Resuscitation Requirements and Renal Injury Post-REBOA Ischemia-Reperfusion Injury

Resuscitative endovascular balloon occlusion of the aorta (REBOA) is a promising treatment for non-compressible torso hemorrhage (NCTH). However, REBOA-associated complications, specifically ischemia-reperfusion injury, significantly limit its therapeutic benefits and broad acceptance.

GJA1-20k is a stress response protein that protects mitochondrial function. Given the central role of deranged cellular metabolism after shock, it was hypothesized that administration of GJA1-20k in a pig model of hemorrhagic shock and REBOA would mitigate REBOA-associated ischemia-reperfusion injury as evidenced by lower serum lactate concentrations.

### Methods

Twelve Yorkshire pigs were anesthetized and underwent a splenectomy. Animals were subjected to hemorrhagic shock by removing 25% of the estimated blood volume, followed by 45 minutes of complete supra-coeliac REBOA. Twenty-five minutes into the aortic occlusion period, animals received a one-hour intravenous infusion of either 0.9% saline (placebo) or 0.01 mg/kg GJA1-20k peptide (SEQ ID NO: 3) from T85 to T145. Animals were transfused with autologous blood, and the balloon catheter was deflated and removed. Lastly, pigs received critical care and algorithmic resuscitation with isotonic crystalloids and norepinephrine to maintain normotension (mean arterial pressure >65 mmHg) for 4.25 hours. The total time of the experiment was six hours (360 minutes). Serum assessments of biomarker expression were performed at different time points throughout the study.

### Results

While the pigs treated with GJA1-20k peptide did exhibit slightly lower serum lactate concentrations as compared to placebo-treated control pigs (Control: 5.6 [3.0-6.7] mmol/L, GJA1-20k: 3.5 [3.0-3.8] mmol/L), this effect did not reach statistical significance (p=0.1). However, the lack of a statistically significant difference between the treatment groups may have been due to the large volume of fluids received in the control group versus the GJA1-20k treatment group, or due to this initial study being underpowered. Additional studies including at least 10 pigs per treatment group will determine whether there is a statistically significant difference in serum lactate concentration levels between pigs treated with GJA1-20k peptide and control (assuming a two-tailed t-test and a power of 80% with an alpha of 0.05).

A significant reduction in resuscitation IV fluid requirements was demonstrated for the GJA1-20k peptide treatment group (Control: 62.5 [50-90] mL/kg, GJA1-20k: 22.5 [0-50] mL/kg, p=0.03). Importantly, with GJA1-20k treatment, two animals required no fluid boluses, and another animal only required 10 mL/kg of isotonic crystalloids after aortic occlusion and balloon deflation. GJA1-20k treatment was also associated with a significant reduction in final serum creatinine concentration (Control: 2.8±0.3 mg/dl, GJA1-20k: 2.4+0.3 mg/dl, p=0.03). Further, there was no significant difference in urine output (Control: 10.8 [8.7-12.5] mL/kg; GJA1-20k: 14.8 [8.8-21.3] mL/kg; p=0.48), but the total fluid balance [Fluid bolus (mL/kg)-Urine output (mL/kg)] was in favor of less fluid overload in the GJA1-20k-treated pigs (Control: 53.3 [41.3-79.8] ml/kg; GJA1-20k: 18.8 [1.2-34.4] ml/kg; p=0.04). These numbers must be interpreted in light of the fact that pigs treated with GJA1-20k received significantly less fluids than the control pigs. Therefore, the renal protective effects of GJA1-20k are likely of an even larger magnitude than was observed in this study (e.g., the creatinine would be even lower and the urine output higher with some fluids).

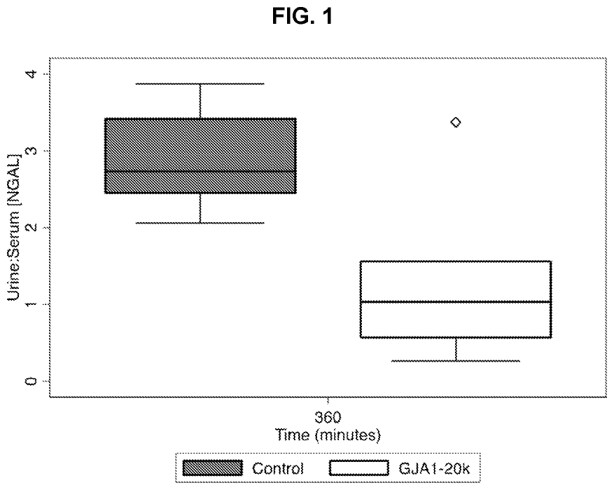

As shown in FIG. 1, the urine-to-serum concentration level of neutrophil gelatinase-associated lipocalin (NGAL) was lower in GJA1-20k peptide-treated pigs as compared to the control-treated pigs at the end of the six-hour experiment. NGAL is an early biomarker of renal injury and higher levels of NGAL in urine are associated with renal injury from conditions such as ischemia-reperfusion. Therefore, these results demonstrated that GJA1-20k treatment mitigated renal injury post-REBOA.

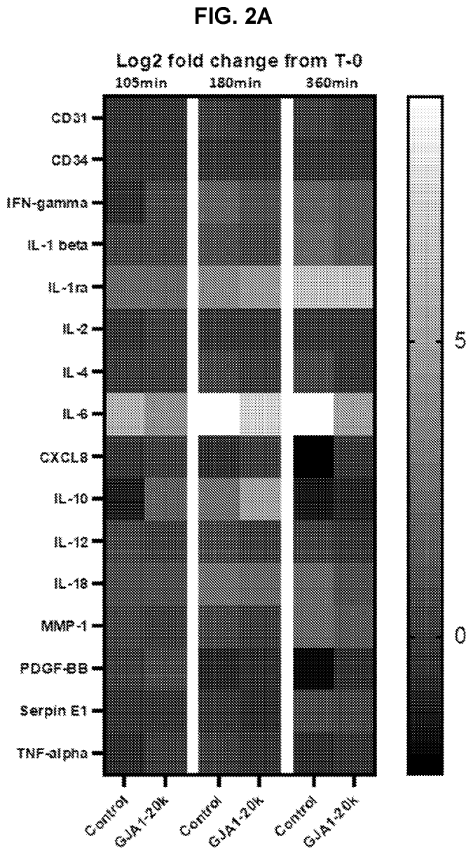

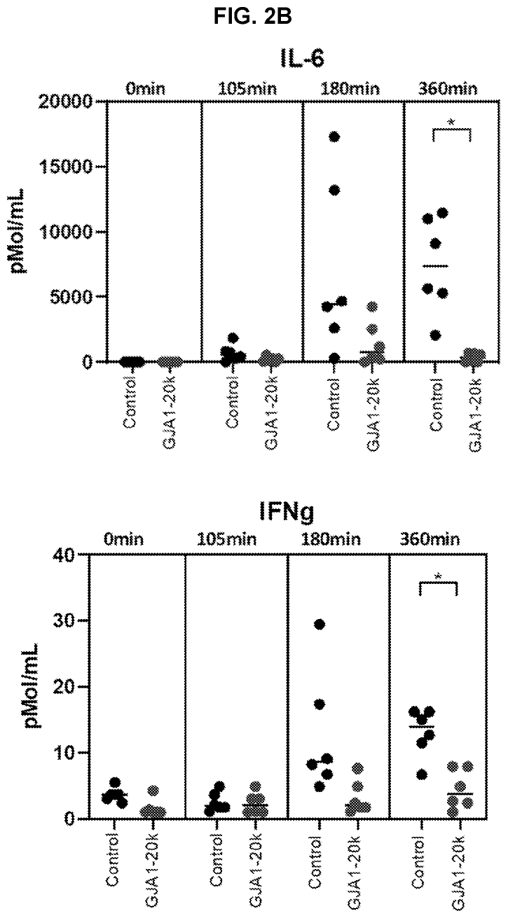

Furthermore, FIG. 2A-B show the serum concentrations of various cytokines over time in control-treated and GJA1-20k peptide-treated pigs. FIG. 2A shows a heatmap of the log 2 fold change for serum protein expression levels of 16 different cytokines in control-treated and GJA1-20k peptide-treated pigs at 105 min, 180 min, and 360 min from baseline T-0. FIG. 2B shows the serum concentration levels (pMol/mL) for IL-6 (top) and IFNg (bottom) in control-treated and GJA1-20k peptide-treated pigs at 0 min, 105 min, 180 min, and 360 min. Higher serum levels of these cytokines are associated with tissue injury and are directly related to mitochondrial injury. GJA1-20k treatment resulted in significant decreases in the serum levels of both IL-6 and IFNg at 6 hours (FIG. 2B). Therefore, these data further suggested that GJA1-20k treatment reduced mitochondrial dysfunction and protected pigs against ischemia-reperfusion injury.

The observed resuscitation requirements in the GJA1-20k treatment group were extremely low considering the severity of ischemia-reperfusion injury in this REBOA model and the median volume of isotonic crystalloids that the pigs in the control group received. GJA1-20k treatment significantly reduced IV fluid resuscitation requirements and mitigated renal injury following REBOA in this pig model of hemorrhagic shock. Mitochondrial protection by GJA1-20k is a potential target for the mitigation of REBOA-associated ischemia-reperfusion injury after hemorrhagic shock.

1. A method of treating, preventing, reducing the likelihood of having, reducing the severity of, and/or slowing the progression of ischemia-reperfusion injury in a subject, the method comprising:
administering to the subject a therapeutically effective amount of a pharmaceutical composition comprising:
a GJA1-20k polypeptide or functional variant or fragment thereof; or
a GJA1-20k gene expression vector comprising a polynucleotide sequence encoding a GJA1-20k polypeptide or functional variant or fragment thereof.

2. The method of claim 1, wherein the pharmaceutical composition is administered to the subject by intravenous (IV) injection.

3. The method of claim 1, wherein the GJA1-20k polypeptide or functional variant or fragment thereof comprises an amino acid sequence having at least 90-99% identity to any one of SEQ ID NO: 1 or 3, or an amino acid sequence that is encoded by a polynucleotide having 90-99% identity to SEQ ID NO: 4.

4. The method of claim 1, wherein the GJA1-20k polypeptide or functional variant or fragment thereof comprises an amino acid sequence selected from any one of SEQ ID NO: 1 or 3, or an amino acid sequence that is encoded by SEQ ID NO: 4.

5. The method of claim 1, wherein the GJA1-20k polypeptide or functional variant or fragment thereof further comprises a polyaspartate (D10) peptide tag (SEQ ID NO: 2).

6-7. (canceled)

8. The method of claim 1, wherein the GJA1-20k gene expression vector is selected from a viral vector, an adeno-associated virus (AAV) vector, a recombinant AAV (rAAV) vector, a single-stranded AAV vector, a double-stranded AAV vector, a self-complementary AAV (scAAV) vector, or combinations thereof.

9. The method of claim 8, wherein the GJA1-20k gene expression vector is an AAV vector of a serotype selected from AAV1, AAV2, AAV3, AAV4, AAV5, AAV6, AAV7, AAV8, AAV9, AAV10, AAV11, AAV12, or a hybrid serotype thereof.

10. The method of claim 9, wherein the GJA1-20k gene expression vector is an AAV9 vector.

11. The method of claim 1, wherein the ischemia-reperfusion injury in the subject is the result of severe bleeding, hemorrhagic or hypovolemic shock, resuscitative endovascular balloon occlusion of the aorta, solid organ transplant, cardiac bypass surgery, cardiac angioplasty, radio-opaque dye injury to kidneys, damage to downstream organs in vascular surgery, or combinations thereof.

12. A pharmaceutical composition comprising a GJA1-20k polypeptide or functional variant or fragment thereof.

13. The pharmaceutical composition of claim 12, wherein the GJA1-20k polypeptide or functional variant or fragment thereof comprises an amino acid sequence having at least 90-99% identity to any one of SEQ ID NO: 1 or 3.

14. The pharmaceutical composition of claim 12, wherein the GJA1-20k polypeptide or functional variant or fragment thereof comprises an amino acid sequence selected from any one of SEQ ID NO: 1 or 3.

15. The pharmaceutical composition of claim 12, wherein the GJA1-20k polypeptide or functional variant or fragment thereof further comprises a polyaspartate (D10) peptide tag (SEQ ID NO: 2).

16. A pharmaceutical composition comprising a GJA1-20k gene expression vector comprising a polynucleotide sequence encoding a GJA1-20k polypeptide or functional variant or fragment thereof.

17. The pharmaceutical composition of claim 16, wherein the polynucleotide sequence has at least 90-99% identity to SEQ ID NO: 4.

18. The pharmaceutical composition of claim 16, wherein the polynucleotide sequence is SEQ ID NO: 4.

19. The pharmaceutical composition of claim 16, wherein the GJA1-20k gene expression vector is selected from a viral vector, an adeno-associated virus (AAV) vector, a recombinant AAV (rAAV) vector, a single-stranded AAV vector, a double-stranded AAV vector, a self-complementary AAV (scAAV) vector, or combinations thereof.

20. The pharmaceutical composition of claim 19, wherein the GJA1-20k gene expression vector is an AAV vector of a serotype selected from AAV1, AAV2, AAV3, AAV4, AAV5, AAV6, AAV7, AAV8, AAV9, AAV10, AAV11, AAV12, or a hybrid serotype thereof.

21. The pharmaceutical composition of claim 20, wherein the GJA1-20k gene expression vector is an AAV9 vector.

22-25. (canceled)


SEQUENCE LISTING (4 sequences)

SEQ ID NO: 1 - protein, 170 aa, Homo sapiens
MLVVSLVSLALNIIELFYVFFKGVKDRVKGKSDPYHATSGALSPAKDCGSQKYAYFNGCSSPTAPLSPMSPPGYKLVTGDRNNSSCRNYNKQASEQNWANYSAEQNRMGQAGSTISNSHAQPEDFPDDNQNSKKLAAGHELQPLAIVDQRPSSRASSRASSRPRPDDLEI

SEQ ID NO: 2 - protein, 10 aa, synthetic construct
DDDDDDDDDD

SEQ ID NO: 3 - protein, 180 aa, synthetic construct
MHHHHHHGGGGSGGGGSVKDRVKGKSDPYHATSGALSPAKDCGSQKYAYFNGCSSPTAPLSPMSPPGYKLVTGDRNNSSCRNYNKQASEQNWANYSAEQNRMGQAGSTISNSHAQPFDFPDDNQNSKKLAAGHELQPLAIVDQRPSSRASSRASSRPRPDDLEIGGSGGSDDDDDDDDDD

SEQ ID NO: 4 - DNA, 534 bp, synthetic construct
  source 1..534: mol_type = other DNA
atgcatcatcaccatcaccacatgctggtggtgtccttggtgtccctggccttgaatatcattgaactcttctatgttttcttcaagggcgttaaggatcgggttaagggaaagagcgacccttaccatgcgaccagtggtgcgctgagccctgccaaagactgtgggtctcaaaaatatgcttatttcaatggctgctcctcaccaaccgctcccctctcgcctctatctcctcctgggtacaagctggttactggcgacagaaacaattcttcttgccgcaattacaacaagcaagcaagtgagcaaaactgggctaattacagtgcagaacaaaatcgactagggcaggcgggaagcaccatctctaactcccatgcacagccttttgatttccccgatgataaccagaattctaaaaaactagctgctggacatgaattacagccactagccattgtggaccagcgaccttcaagcagagccagcagtcgtgccagcagcagacctcggcctgatgacctggagatctag